(19) United States
(12) Patent Application Publication
(10) Pub. No.: US 2013/0158413 A1
(43) Pub. Date: Jun. 20, 2013

(54) OPTICAL MEASUREMENT OF PHYSIOLOGICAL BLOOD PARAMETERS

(71) Applicants: Daniel Lisogurski, Boulder, CO (US); Friso Schlottau, Lyons, CO (US); Lockett Wood, Lyons, CO (US)

(72) Inventors: Daniel Lisogurski, Boulder, CO (US); Friso Schlottau, Lyons, CO (US); Lockett Wood, Lyons, CO (US)

(73) Assignee: Nellcor Puritan Bennett LLC, Boulder, CO (US)

(21) Appl. No.: 13/327,384

(22) Filed: Dec. 15, 2011

(51) Int. Cl. A61B 6/00

(52) U.S. Cl. 600/476

(57) ABSTRACT

Systems and methods for measuring a physiological parameter of tissue in a patient are provided herein, such as a system to optically analyze tissue of a patient. An example system includes a tissue interface assembly configured to emit an input optical signal into the tissue, receive a reference optical signal and a measurement optical signal from the tissue, and transfer the reference optical signal and the measurement optical signal to the optical link. The optical link is configured to transfer the reference optical signal and the measurement optical signal. The transceiver is configured to receive and convert the optical signals into digital signals.

### TECHNICAL FIELD

Aspects of the disclosure are related to the field of medical devices, and in particular, enhancement in the optical measurement of physiological parameters of blood and tissue.

### TECHNICAL BACKGROUND

Various devices, such as pulse oximetry devices, can measure some parameters of blood flow in a patient, such as heart rate and oxygen saturation of hemoglobin. Pulse oximetry devices are a non-invasive measurement device, typically employing solid-state lighting elements, such as light-emitting diodes (LEDs) or solid state lasers, to introduce light into the tissue of a patient. The light is then detected and analyzed to determine the parameters of the blood flow in the patient. However, conventional pulse oximetry devices typically only measure certain blood parameters, and are subject to patient-specific noise and inconsistencies which limits the accuracy of such devices.

Photon Density Wave (PDW) techniques can improve on conventional pulse oximetry devices by allowing for measurement of additional physiological parameters. In PDW techniques, high-frequency modulated optical signals are emitted into tissue of a patient. These modulated optical signals are then detected through the tissue and subsequently analyzed to identify physiological parameters such as the heart rate and the oxygen saturation of hemoglobin.

In many examples of PDW measurement, the measurement and processing systems are located remotely from various optical elements used for interfacing optical signals with the tissue of the patient. This configuration can provide some patient mobility by using a flexible fiber optic cable between the equipment. However, having a long cable can introduce errors into the measurement and subsequent processing of the optical signals. Furthermore, interfacing optical elements with tissue can pose problems for repeatability and consistency of measurements.

### OVERVIEW

Systems and methods for measuring a physiological parameter of tissue in a patient are provided herein. In a first example, a system to optically analyze tissue of a patient is provided. The system includes a tissue interface assembly configured to emit an input optical signal into the tissue, receive a reference optical signal and a measurement optical signal from the tissue, and transfer the reference optical signal and the measurement optical signal to the optical link. The optical link is configured to transfer the reference optical signal and the measurement optical signal. The transceiver is configured to receive and convert the reference optical signal into a digital reference signal and to receive and convert the measurement optical signal into a digital measurement signal.

In another example, a system to optically analyze tissue of a patient is provided. The system includes a tissue interface assembly configured emit an input optical signal at a first wavelength and modulated at a first frequency into the tissue, receive a reference optical signal and a measurement optical signal from the tissue, and transfer the reference optical signal and the measurement optical signal to the fiber optic cable. The fiber optic cable comprises a second optical fiber configured to transfer the reference optical signal and the measurement optical signal from the tissue interface assembly to the transceiver. The transceiver is configured to receive and convert the reference optical signal into a digital reference signal and to receive and convert the measurement optical signal into a digital measurement signal. The system also includes a digital processor configured to process the digital measurement signal and the digital reference signal to determine a phase delay between the digital measurement signal and the digital reference signal and process the phase delay to determine physiological parameter for the tissue.

In another example, a method of operating a system to analyze tissue of a patient is provided. The method includes generating an input optical signal and transferring the input optical signal over a fiber optic cable, and receiving the input optical signal from the fiber optic cable and emitting the input optical signal into the tissue. The method also includes receiving a reference optical signal and a measurement optical signal from the tissue and transferring the reference optical signal and the measurement optical signal over the fiber optic cable, receiving the reference optical signal and the measurement optical signal from the fiber optic cable and converting the reference optical signal into a digital reference signal and converting the measurement optical signal into a digital measurement signal. The method also includes processing the digital measurement signal and the digital reference signal to determine a phase delay between the digital measurement signal and the digital reference signal and processing the phase delay to determine physiological parameter for the tissue.

### BRIEF DESCRIPTION OF THE DRAWINGS

FIG. 1 is a system diagram illustrating a system for measuring a physiological parameter of blood in a patient.

FIG. 2 is a system diagram illustrating a system for measuring a physiological parameter of blood in a patient including a tissue interface assembly.

FIG. 3 is a system diagram illustrating a system for measuring a physiological parameter of blood in a patient including a tissue interface assembly.

FIG. 4 is a system diagram illustrating a system for measuring a physiological parameter of blood in a patient including a tissue interface assembly.

FIG. 5 is a system diagram illustrating a tissue interface assembly.

FIG. 6 is a system diagram illustrating a tissue interface assembly.

FIG. 7 is a system diagram illustrating a tissue interface assembly.

FIG. 8 is an oblique view diagram illustrating a tissue interface pad.

FIG. 9 is a system diagram illustrating a measurement environment for measuring a physiological parameter of blood in a patient.

FIG. 10 is a flow diagram illustrating a method of operation of a system for measuring a physiological parameter of blood in a patient.

### DETAILED DESCRIPTION

Various physiological parameters of tissue and blood of a patient can be determined non-invasively, such as optically. In one example, optical signals introduced into the tissue of the patient are modulated according to a high-frequency modulation signal to create a photon density wave (PDW) optical signal in the tissue undergoing measurement. Due to the interaction between the tissue or blood and the PDW optical signal, various characteristics of the PDW optical signal can be affected, such as through scattering or propagation by various components of the tissue and blood.

For example, a phase delay or amplitude of optical signals could be identified. A phase delay of a PDW optical signal is sensitive to changes in the scattering properties or scattering coefficient of the measured tissue, whereas the amplitude of a PDW optical signal is sensitive to concentrations of an absorber in the measured tissue or to an absorption coefficient. Tissue beds are typically approximated as a homogenous mixture of blood and other tissues containing no blood. In general terms, the ratio of the differentials of the PDW amplitudes to the phase delay signals is a linear function of the absorption coefficient of the probed tissue, and can be used to derive a total hemoglobin concentration (tHb) measurement. Other physiological parameters could be determined, and these physiological parameters could include any parameter associated with the blood or tissue of the patient, such as regional oxygen saturation (rSO2), arterial oxygen saturation (SpO2), heart rate, lipid concentrations, among other parameters, including combinations thereof.

Although the term ‘optical’ or ‘light’ is used herein for convenience, it should be understood that the applied and detected signals are not limited to visible light, and could comprise any photonic, electromagnetic, or energy signals, such as visible, infrared, ultraviolet, radio, x-ray, gamma, or other signals. Additionally, the use of optical fibers or optical cables herein is merely representative of a waveguide used for propagating signals between a transceiver and tissue of a patient. Suitable waveguides would be employed for different electromagnetic signal types.

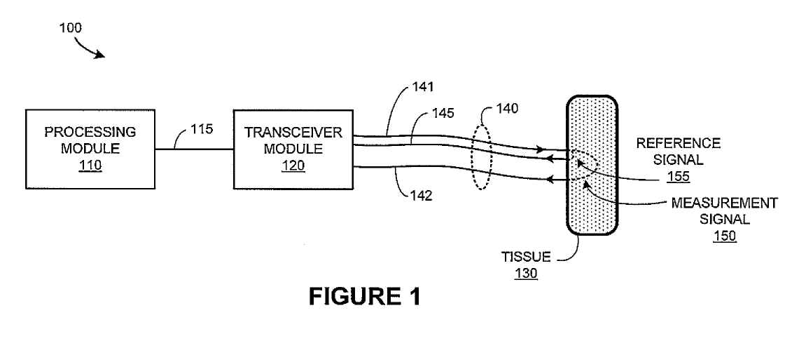

As a first example of a system for measuring a physiological parameter of blood in a patient, FIG. 1 is presented. FIG. 1 illustrates system 100, which includes processing module 110, transceiver module 120, and tissue 130. Processing module 110 and transceiver module 120 communicate over link 115. Transceiver module 120 emits and receives optical signals over optical links, namely optical links 141, 142, and 145. In some examples, optical links 141, 142, and 145 could comprise optical fibers and be included in a composite link or cable, such as indicated by optical link 140. It should be understood that separate or combined physical links could be employed.

In FIG. 1, tissue 130 comprises tissue of a patient, such as a finger, toe, arm, leg, earlobe, forehead, or other tissue portion of a patient. Tissue 130 is a portion of the tissue of a patient undergoing measurement of a physiological blood parameter, and is represented by a generally rectangular element for simplicity herein. The wavelength of signals applied to the tissue can be selected based on many factors, such as optimized to a wavelength strongly absorbed by hemoglobin, lipids, proteins, or other tissue and blood components of tissue 130.

In operation, optical signals are applied to tissue 130 for measurement of a physiological parameter, as indicated by measurement signal 150 and reference signal 155. In this example, both measurement signal 150 and reference signal 155 are applied to tissue 130 over link 141, and comprise the same input optical signal. Each of links 142 and 145 then receive optical signals which have been propagated, reflected, or scattered by tissue 130.

As shown in FIG. 1, reference link 145 is positioned proximate to input link 141 at a first location, and measurement link 142 is positioned further away than reference link 145 at a second location or distance from input link 141. Thus, measurement signal 150 will include optical energy which has undergone more propagation through tissue 130 than reference signal 155. More specifically, the optical signals received as reference signal 155 are typically reflected or scattered from tissue 130 without significant penetration. Likewise, the optical signals received as measurement signal 150 are typically reflected or scattered from the tissue with significant tissue penetration. This amount of penetration is roughly indicated by the dashed lines included in tissue 130. In further examples, the optical signals transported by input link 141 are coupled through an interface element to reference link 145, and thus reference link 145 would not rely on tissue propagation.

Advantageously, note the similarity in the physical paths taken by the optical signals traversing input link 141 and reference link 145, and the difference in propagation by the optical signals traversing tissue 130. With system 100, the dominant path difference between reference signal 145 and measurement signal 142 now occurs via tissue 130. Thus, errors or inaccuracies that would be introduced by using different physical paths are largely mitigated, and detection of differences in optical signals detected from measurement signal 150 and reference signal 155 through tissue 130 is enhanced.

More specifically, a phase measurement of the example in FIG. 1 is more accurate than a phase measurement of a system which compares only an optical measurement signal against an electrical reference used to drive a light source. The phase difference when an electrical reference is used is limited by errors in an optical path through tissue as well as an optical path through the entire measurement system including any optical fibers. Bending optical fibers may change the path length and introduce errors. Thus, in this example, a reference signal travels with input link 141, such as when packaged together in a cable bundle, and has essentially the same bends as input link 141 and measurement link 142. Any phase changes between the associated reference and measurement signals are almost entirely due to the path of light through the tissue instead of system and length-introduced errors.

Upon receiving optical signals over links 142 and 145, transceiver module 120 in combination with processing module 110 will process the detected optical signals to determine various characteristics of the detected optical signals. Physiological parameters of the tissue and patient can then be identified based on the various characteristics of the detected optical signals.

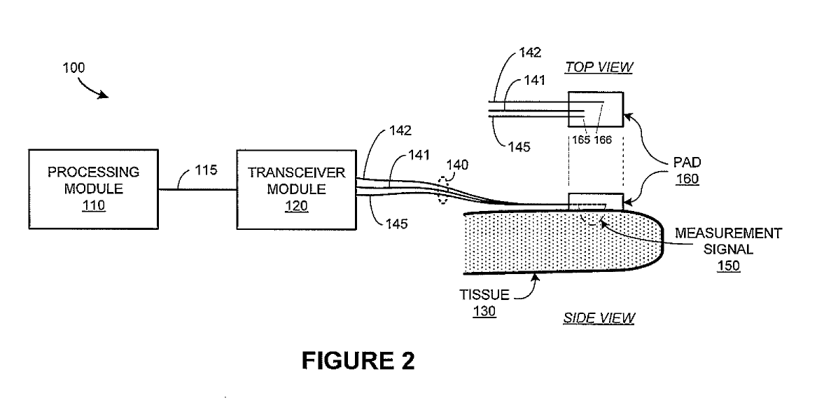

FIG. 2 is a system diagram illustrating further configuration of system 100 for measuring a physiological parameter of blood in a patient. FIG. 2 includes similar elements as FIG. 1, but also includes a tissue interface assembly comprising pad 160. A top view and a side view of pad 160 are included in FIG. 2 for clarity. Each of optical links 141, 142, and 145 are disposed partially within pad 160.

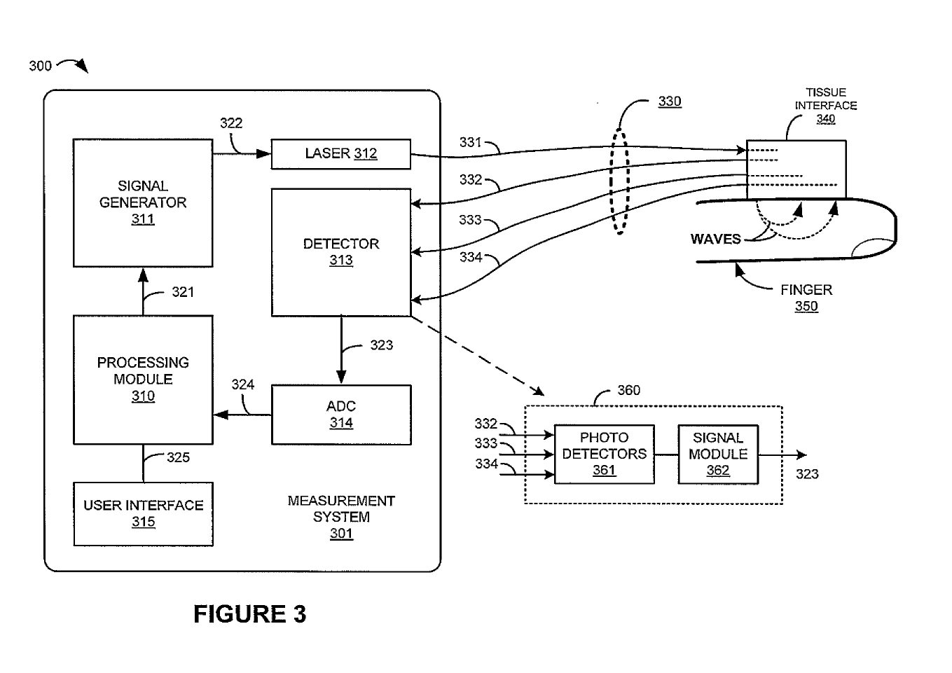

Pad 160 comprises a physical structure having a surface that couples to biological tissue, namely tissue 130. The surface comprises at least one optical signal emission point and at least one optical signal detection point. Pad 160 includes a mechanical arrangement to position and hold each of optical links 141, 142, and 145 in a generally parallel arrangement to one another and to tissue 130. These mechanical arrangements could include grooves, c-grooves, channels, holes, snap-fit features, or other elements to route the associated optical link, such as optical fiber, to a desired position on pad 160. As shown in FIG. 2, pad 160 positions an end of input optical link 141 at location 165, and end of reference link 145 also at location 165, and an end of measurement optical link 142 at location 166. Due to the arrangement of the side view in FIG. 2, only measurement signal 150 is shown in tissue 130 and reference signal 155 is excluded for clarity. Pad 160 may be comprised of plastic, foam, rubber, glass, metal, adhesive, or some other material, including combinations thereof. Further examples of pad 160 are included in FIGS. 3-8 and discussed herein.

Referring back to FIGS. 1 and 2, processing module 110 comprises communication interfaces, digital processors, computer systems, microprocessors, circuitry, non-transient computer-readable media, user interfaces, or other processing devices or software systems, and may be distributed among multiple processing devices. Processing module 110 could be included in the equipment or systems of transceiver module 120, or could be included in separate equipment or systems. Examples of processing module 110 may also include software such as an operating system, logs, utilities, drivers, databases, data structures, processing algorithms, networking software, and other software stored on a non-transient computer-readable medium.

Transceiver module 120 comprises electrical to optical conversion circuitry and equipment, optical modulation equipment, and optical waveguide interface equipment. Transceiver module 120 could include direct digital synthesis (DDS) components, laser driver components, CD/DVD laser driver circuitry, function generators, oscillators, or other signal generation components, filters, delay elements, signal conditioning components, such as passive signal conditioning devices, attenuators, filters, and directional couplers, active signal conditioning devices, amplifiers, or frequency converters, including combinations thereof. Transceiver module 120 could also include switching, multiplexing, or buffering circuitry, such as solid-state switches, RF switches, diodes, or other solid state devices. Transceiver module 120 also includes laser elements such as a laser diode, solid-state laser, or other laser device, along with associated driving circuitry. Optical couplers, cabling, or attachments could be included to optically mate laser elements to links 141, 142, and 145. Transceiver module 120 also comprises light detection equipment, optical to electrical conversion circuitry, photon density wave characteristic detection equipment, and analog-to-digital conversion equipment. Transceiver module 120 could include a photodiode, phototransistor, photomultiplier tube, avalanche photodiode (APD), or other optoelectronic sensor, along with associated receiver circuitry such as amplifiers or filters. Transceiver module 120 could also include phase and amplitude detection circuitry, mixers, oscillators, or other signal detection and processing elements.

Tissue 130 comprises a portion of the tissue of a patient undergoing measurement of a physiological blood parameter. It should be understood that tissue 130 could represent a finger, fingertip, toe, earlobe, forehead, or other tissue portion of a patient undergoing physiological parameter measurement. Tissue 130 could comprise muscle, fat, blood, vessels, or other tissue components. The blood portion of tissue 130 could include tissue diffuse blood and arterial or venous blood. In some examples, tissue 130 is a test sample or representative material for calibration or testing of system 100, such as a piece of Teflon.

Optical links 141, 142, and 145 each comprise an optical waveguide, and use glass, polymer, air, space, or some other material as the transport media for transmission of light, and could each include multimode fiber (MMF) or single mode fiber (SMF) materials. A sheath or loom could be employed to bundle each of optical links 141, 142, and 145 together for convenience as indicated by link 140. One end of each of optical links 141, 142, and 145 mates with an associated component of system 100, and the other end of each of optical links 141, 142, and 145 is configured to optically interface with tissue 130. Various optical interfacing elements could be employed to optically couple links 141, 142, and 145 to tissue 130.

Link 115 uses metal, glass, optical, air, space, or some other material as the transport media, and comprises analog, digital, RF, optical, or power signals, including combinations thereof. Link 115 could use various communication protocols or formats, such as Controller Area Network (CAN) bus, Inter-Integrated Circuit (I2C), 1-Wire, Radio Frequency Identification (RFID), optical, circuit-switched, Internet Protocol (IP), Ethernet, wireless, Bluetooth, communication signaling, or some other communication format, including combinations, improvements, or variations thereof. Link 115 could be a direct link or may include intermediate networks, systems, or devices, and could include a logical network link transported over multiple physical links.

Links 115, 141, 142, and 145 may each include many different signals sharing the same associated link, as represented by the associated lines in FIGS. 1 and 2, comprising channels, forward links, reverse links, user communications, overhead communications, frequencies, wavelengths, modulation frequencies, carriers, timeslots, spreading codes, logical transportation links, packets, or communication directions.

Note that optical link 141 in FIG. 1 could be replaced with an electrical link such as a coaxial cable, where the electrical link could include electrical signaling for driving a laser source or other optical elements. In these examples, an optical emitter could be coupled to tissue 130 for emitting optical signals into tissue 130 in response to the signals of the electrical link. Reference link 145 and measurement link 142 would then carry optical signals received from tissue 130. In yet further examples, link 141 could be an optical link while links 142 and 145 include electrical links coupled to detection elements on tissue 130. In yet further examples, a reverse configuration could be employed, where links 142 and 145 could be coupled to optical sources and link 141 could be coupled to a detector.

Also, although FIGS. 1 and 2 illustrate only a single optical input link 141 and a single measurement link 142, it should be understood that any number of input links and measurement links could be included, as well as any associated optical source and detector equipment. For example, system 100 may have two optical signal sources at different physical locations on tissue 130, which could comprise different wavelengths. Alternatively, or in addition, system 100 may have multiple measurement links at different distances from any input links or over different anatomical structures. However, any reference signals are typically located proximate to an associated input link.

FIG. 3 is a system diagram illustrating system 300 which includes measurement system 301 and tissue interface 340 for measuring a physiological parameter of blood in a patient. In FIG. 3, one input link is employed in combination with multiple output or measurement links. Measurement system 301 and tissue interface 340 are coupled via optical cable 330 which includes several optical links, namely links 331-334. FIG. 3 also includes finger 350 of a patient undergoing measurement of a physiological parameter. A portion of finger 350 is shown far clarity, and the finger could instead be a different portion of the tissue of a patient. Finger 350 comprises tissue components such as blood, capillaries, arteries, veins, fat, muscle, bone, nails, or other biological tissue and associated components.

Measurement system 301 includes components and equipment to emit optical signals into finger 350, detect the optical signals propagated through tissue of finger 350, and process characteristics of optical signals for determination of physiological parameters. Measurement system 301 includes processing module 310, signal generator 311, laser 312, detector 313, analog-to-digital converter (ADC) 314, and user interface 315. These individual modules will be discussed below. A transceiver portion of measurement system 301 could comprise signal generator 311, laser 312, detector 313, analog-to-digital converter (ADC) 314, although different elements could be included.

Tissue interface 340 is configured to couple with finger 350 and provide optical mating between optical links 331-334 and tissue of finger 350. Further elements could be included in tissue interface 340, such as a clamp, spring, band, adhesive, elastic sleeve, or other elements to couple tissue interface 340 physically to finger 350. Tissue interface 340 may be comprised of plastic, foam, rubber, glass, metal, adhesive, or some other material, including combinations thereof.

Optical cable 330 includes individual signaling links in this example, namely links 331-334. Also in this example, link 331 is an input optical link, link 332 is a reference optical link, link 333 is a first measurement optical link, and link 334 is a second measurement optical link. Each of links 331-334 could comprise individual optical fibers. Optical cable 330 could include a sheath or loom to bundle each of links 331-334 together for convenience. One end of each of optical links 331-334 terminates and optically mates with an associated component of measurement system 301, and the other end of each of optical links 331-334 is configured to terminate in tissue interface 340 and interface optically with finger 350.

In operation, tissue interface 340 will be coupled to finger 350 of a patient undergoing measurement of physiological parameters. Although tissue interface 340 is shown on the underside of finger 350 (as indicated by the fingernail position), tissue interface 340 could be applied to any portion of finger 350. A user will instruct through user interface 315 to initiate a measurement process with measurement system 301. These user instructions will be transferred over link 325 for receipt by processing module 310. In response, processing module 310 will initiate control signaling over link 321 to instruct signal generator 311 to generate signals for laser 312. Laser 312 will emit optical signals on optical link 331 according to the input received over link 322. In this example, link 322 is employs electrical signaling and laser 312 outputs optical signals over link 331 according to the electrical signaling.

In some examples, photon density wave (PDW) techniques are employed within finger 350. To establish a PDW, signal generator 311 first generates a high-frequency modulated drive signal for laser 312. This high-frequency modulated signal could comprise an amplitude modulated signal at one gigahertz or higher. It should be understood that lower modulation frequencies could be employed. Laser 312 receives this modulated signal over link 322 and in response, emits a corresponding optical signal modulated according to the received modulated signal. Thus, although laser 312 emits an optical signal of a certain wavelength, this optical signal is further modulated at a high rate, according to the received signal over link 322. In some examples, a solid-state switch element could be employed in signal generator 311 to modulate the input signal for laser 312, while in other examples, an optical switch could be employed on the output of laser 312 to modulate the optical signal according to the high-frequency modulation signal.

In tissue interface 340, the optical links are shown routed to varying locations indicated by the dashed hidden lines. Input link 331 is routed to a first location, reference link 332 is routed to a similar location as input link 331, first measurement link 333 is routed to a second location, and second measurement link 334 is routed to a third location. Accordingly, input link 331 will have an emission point for an optical signal at the location shown. Each of first measurement link 333 and second measurement link 334 will receive the optical signal at their respective locations, as indicated by the “waves” arrows in FIG. 3. It should be noted that the routes and depths shown for the links in tissue interface 340 are merely exemplary, and the vertical stacked configuration is used to emphasize the depth of routing for each link, not to imply a vertically stacked routing in tissue interface 340.

Since the termination point of reference link 332 is located adjacent or proximate to the termination point of input link 331, any optical signal emitted by input link 331 would only propagate a short distance for receipt into reference link 332. In examples where separate optical fibers are employed, the optical fiber associated with input link 331 and the optical fiber associated with reference link 332 would terminate at the same or similar location within tissue interface 340. Likewise, any optical signal received by first measurement link 333 or second measurement link 334 would have propagated through a deeper and more substantial portion of finger 350 than optical signals detected by reference link 332.

The optical signals received by each of links 332-334 is transferred over optical cable 330 for receipt by detector 313. Detector 313 includes optical detection elements which convert the received optical signals to corresponding analog electrical signals. Detector 313 could also include elements to determine characteristics of the optical signals, such as amplitude, intensity, or phase delays. Phase delay detection elements could include comparing the optical signals received over first measurement link 333 and second measurement link 334 to the optical signal received over reference link 332. Filters could be employed to discriminate the optical signals or desired characteristics from other optical energy or electrical noise. ADC 314 would then receive over link 323 the electrical signals as determined by detector 313 and convert these signals into a digital format for delivery to processing module 310 over link 324. Processing module 310 processes the received information to determine characteristics of the received signals as well as identify values of physiological parameters based on the received signals, such as the heart rate and the oxygen saturation of hemoglobin. Processing module 310 could transfer these values of the physiological parameters to user interface 315 over link 325 for display to a user.

Alternatively, measurement system 301 may comprise an analog circuit such as an Analog Devices AD8302 to determine an amplitude and/or a phase difference between optical signals received over reference link 332 and optical signals received over first measurement link 333 or second measurement link 334. ADC 314 could then digitize the phase and/or amplitude differences rather than the received signals themselves. Alternatively, a high-speed, all-digital system couple be employed to perform an auto-gain function, and ADC 314 could be omitted by processing high-speed digital signals directly by measuring the jitter/delay of the digital signals.

Advantageously, in FIG. 3, reference link 332 receives optical signals emitted by input link 331 without significant tissue penetration of finger 350. Any phase delays or amplitude changes detected over first measurement link 333 and second measurement link 334 will be dominated by changes introduced by tissue or blood characteristics of finger 350. This configuration minimizes phase delay and amplitude errors introduced by long optical links since reference link 332 is routed along with input link 331, first measurement link 333, and second measurement link 334. A bend in cable 330 that is caused by patient motion or other physical movement would affect input link 331, reference link 332, first measurement link 333, and second measurement link 334 in a similar manner. Thus, comparisons between reference link 332 and first measurement link 333 or second measurement link 334 would tend to compensate for errors introduced by long or bent optical links.

Thus, any signals received from first measurement link 333 or second measurement link 334 takes a similar path as reference link 332 except through finger 350. Since the light coining in to first measurement link 333 and second measurement link 334 is scattered by finger 350, it may be desirable that any optical signals in reference link 332 is also scattered and instead of merely traveling back in a single optical mode, i.e. not substantially scattered. To accomplish this, optical signals for receipt by reference link 332 could either be transported through a small distance of tissue of finger 350 or could be optically coupled to reference link 332 after an optical element which scatters optical signals appropriately.

Referring back to the elements of measurement system 301, processing module 310 retrieves and executes software or other instructions to direct the operations of the other components of measurement system 301, as well as process data received from ADC 314. In this example, processing module 310 comprises a digital processor, such as a digital signal processor (DSP), and could include a non-transitory computer-readable medium such as a disk, integrated circuit, server, flash memory, or some other memory device, and also may be distributed among multiple memory devices. Examples of processing module 310 include DSPs, micro-controllers, field programmable gate arrays (FPGA), or discrete logic, including combinations thereof. In one example, the DSP comprises an Analog Devices Blackfin® device.

Signal generator 311 comprises electronic components for generating signals for use by laser 312, as well as receiving instructions from processing module 310 for generating these signals. Signal generator 311 produces a signal to drive laser 312 to output a proper optical signal, and signal generator 311 instructs laser 312 with parameters such as intensity, amplitude, phase offset, modulation, on/off conditions, or other parameters. Signal generator 311 could comprise a signal synthesizer, such as a direct digital synthesis (DDS) component, laser driver components, function generators, oscillators, or other signal generation components. Signal generator 311 could also include filters, delay elements, or other calibration components. In some examples, where multiple lasers are employed, signal generator 311 could include high-speed solid state switches.

Laser 312 comprises a laser element such as a laser diode, solid-state laser, vertical-cavity surface-emitting laser (VCSEL), or other laser device, along with associated driving circuitry. Laser 312 emits coherent light over an associated optical fiber, such as link 331. In this example, a wavelength of light is associated with laser 312 and likewise link 331. In other examples, multiple lasers and multiple optical fibers are employed to transfer multiple wavelengths of light into tissue of finger 350. In examples with multiple lasers, laser 312 could comprise multiple laser diodes, such as multiple VCSELs packed in a single component package. The wavelength of light could be tuned to hemoglobin absorbency or an isosbestic point of hemoglobin. Specific examples of wavelength include 590 nanometers (nm), 660 nm, or 808 nm, although other wavelengths could be used. Laser 312 may modify an intensity of the associated laser light, or toggle the associated laser light based on an input signal received from signal generator 311. Optical couplers, cabling, or attachments could be included to optically mate laser 312 to link 331. Additionally, a bias signal may be added or mixed into the signals received from signal generator 311, such as adding a “DC” bias for the laser light generation components.

Detector 313 comprises optical detector elements, such as a photodiode, phototransistor, avalanche photodiode (APD), photomultiplier tube, charge coupled device (CCD), CMOS optical sensor, optoelectronic sensor, or other optical signal sensor along with associated receiver circuitry such as amplifiers or filters. Detector 313 could also include phase or amplitude detector circuitry. Detector 313 receives light over associated links 332-334. Optical couplers, cabling, or attachments could be included to optically mate detector 313 to links 332-334. Detector 313 converts the optical signals received over links 332-334 to electrical signals for transfer to ADC 314. Detector 313 could also include circuitry to condition or filter the signals before transfer to ADC 314. It should be noted that although in this example input optical signal 331 only carries a particular emitted wavelength of light, output links 332-334 can carry any received light from tissue of finger 350, which could include multiple wavelengths or stray light from other light sources. Also, multiple detector elements could be employed and could be shared between multiple laser sources, such as when the detector employs modulation or multiplexing techniques, to detect individual optical signals from combined detected optical signals.

An optional example of detector 313, namely detector 360, is shown in FIG. 3. Detector 360 includes photodetectors 361 and signal module 362. Photodetectors 361 may comprise multiple optical detector elements, such as photodiodes. Photodetectors 361 receive optical signals over the associated optical link 332-334, and covert the optical signals into electronic versions of the optical signals. Further processing could be performed in signal module 362, such as intermediate frequency (IF) signal processing, filtering, conditioning, or other signal processing. Signal module 362 would then transfer the processed electrical versions of the optical signals over link 323 to ADC 314.

Analog-to-digital converter (ADC) 314 comprises analog to digital converter circuitry. ADC 314 receives the detected information from detector 313, and digitizes the information, which could include digitizing intensity, amplitude, or phase information of optical signals converted into electrical signals by detector 313. The dynamic range, bit depth, and sampling rate of ADC 314 could be selected based on the signal parameters of the optical signals driven by laser 312, such as to prevent aliasing, clipping, and for reduction in digitization noise. ADC 314 could each be an integrated circuit ADC, or be implemented in discrete components. ADC 314 provides digitized forms of information for receipt by processing module 310.

User interface 315 includes equipment and circuitry to communicate information to a user of measurement system 301. User interface 315 may include any combination of displays and user-accessible controls and may be part of measurement system 301 as shown or could be a separate patient monitor or multi-parameter monitor. When user interface 315 is a separate unit, user interface 315 may include a processing system and may communicate with measurement system 301 over a communication link comprising a serial port, UART, USB, Ethernet, or wireless link such as Bluetooth, Zigbee or WiFi, among other link types. Examples of the equipment to communicate information to the user could include displays, indicator lights, lamps, light-emitting diodes, haptic feedback devices, audible signal transducers, speakers, buzzers, alarms, vibration devices, or other indicator equipment, including combinations thereof. The information could include raw ADC samples, calculated phase and amplitude information for one or more emitter/detector pairs, blood parameter information, waveforms, summarized blood parameter information, graphs, charts, processing status, patient information, or other information. User interface 315 also includes equipment and circuitry for receiving user input and control, such as for beginning, halting, or changing a measurement process or a calibration process. Examples of the equipment and circuitry for receiving user input and control include push buttons, touch screens, selection knobs, dials, switches, actuators, keys, keyboards, pointer devices, microphones, transducers, potentiometers, non-contact sensing circuitry, or other human-interface equipment.

In FIG. 3, links 321-325 each use metal, glass, optical, air, space, or some other material as the transport media, and comprise analog, digital, RF, optical, or power signals, including combinations thereof. Links 321-325 could each use various communication protocols or formats, such as Controller Area Network (CAN) bus, Inter-Integrated Circuit (I2C), 1-Wire, Radio Frequency Identification (RFID), optical, circuit-switched, Internet Protocol (IP), Ethernet, Wireless Fidelity (WiFi), Bluetooth, communication signaling, or some other communication format, including combinations, improvements, or variations thereof. Links 321-325 could each be direct links or may include intermediate networks, systems, or devices, and could each include a logical link transported over multiple physical links.

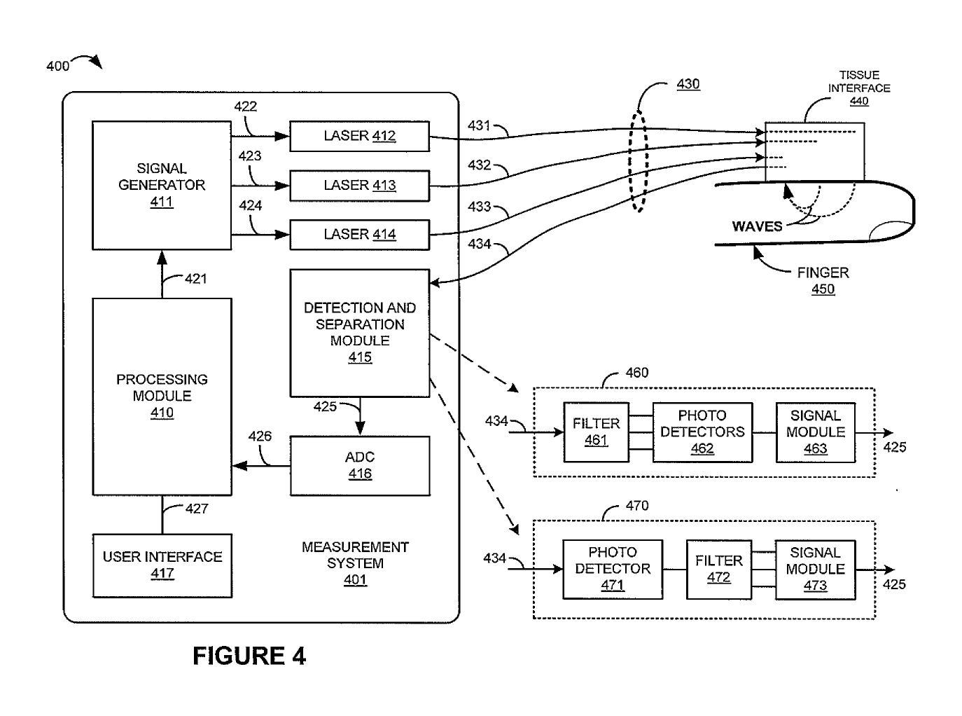

FIG. 4 is a system diagram illustrating system 400 which includes measurement system 401 and tissue interface 440 for measuring a physiological parameter of blood in a patient. In FIG. 4, multiple input signals are employed in combination with a unified output or measurement link. Measurement system 401 and tissue interface 440 are coupled via optical cable 430 which include several optical links, namely links 431-434. FIG. 4 also includes finger 450 of a patient undergoing measurement of a physiological parameter. A portion of finger 450 is shown for clarity, and the finger could instead be a different portion of the tissue of a patient. Finger 450 comprises tissue components such as blood, capillaries, arteries, veins, fat, muscle, bone, nails, or other biological tissue and associated components.

Measurement device 401 includes components and equipment to emit optical signals into finger 450, detect the optical signals as scattered through tissue of finger 450, and process characteristics of the optical signals for determination of physiological parameters. Measurement system 401 includes processing module 410, signal generator 411, lasers 412-414, detection and separation module 415, analog-to-digital converter (ADC) 416, and user interface 417. These individual modules will be discussed below. Processing module 410, signal generator 411, lasers 412-414, detection and separation module 415, ADC 416, user interface 417, and links 421-427 could comprise similar elements, circuitry, equipment, and components as found in similar elements of FIG. 3, although other configurations could be employed. A detailed discussion of the configuration of these elements of FIG. 4 is omitted in light of the discussion above for FIG. 3. A transceiver portion of measurement device 401 could comprise signal generator 411, lasers 412-414, detection and separation module 415, ADC 416, although different elements could be included.

Detection and separation module 415 includes optical or electrical components for detection and separation of signals received over measurement link 434. Detection and separation module 415 could include detection elements as described above for detector 313. In examples where wave division multiplexing (WDM) is employed, detection and separation module 415 includes optical separation elements for separating optical signals of different wavelengths from each other, such as lenses, prisms, optical splitters, optical filters, or other optical separation elements. In examples where frequency division multiplexing (FDM) is employed in PDW modulations, detection and separation module 415 includes electrical signal separation elements, such as filters, bandpass filters, amplifiers, comparators, or other electrical signal separation elements.

In an optional example of detection and separation module 415, detection and separation module 460 is shown in FIG. 4. Detection and separation module 460 could be employed in WDM examples. Detection and separation module 460 includes filter 461, photodetectors 462, and signal module 463. Filter 461 comprises optical filters, such as optical separation elements to separate a composite optical signal into individual optical signals based on wavelength. Filter 461 would receive a composite optical signal over link 434 comprising multiple wavelengths. Filter 461 would then separate the composite optical signal into separate optical signals based on wavelength for transfer to photodetectors 462. Photodetectors 462 may comprise multiple optical signal detector elements, such as photodiodes, or could include a single time-shared optical signal detector element. Photodetectors 462 receive optical signals from filter 461, and covert the optical signals into analog electrical versions of the optical signals. Further processing could be performed in signal module 463, such as intermediate frequency (IF) signal processing, filtering, conditioning, or other signal processing. Signal module 463 would then transfer the processed electrical versions of the optical signals over link 425 to ADC 416.

In another optional example of detection and separation module 415, detection and separation module 470 is shown in FIG. 4. Detection and separation module 470 could be employed in FDM examples, where multiple modulation frequencies are employed. Detection and separation module 470 includes photodetector 471, filter 472, and signal module 473. Photodetector 471 comprises optical signal detector elements, such as photodiodes. Photodetector 471 receives optical signals over link 434, and coverts the optical signals into analog electrical versions of the optical signals for transfer to filter 472. Filter 472 comprises electrical signal filters, such as bandpass filters to separate a composite electrical signal into individual electrical signals. Filter 472 would receive a composite electrical signal from photodetector 471 comprising multiple modulation frequencies. Filter 472 would then separate the composite electrical signal into separate electrical signals based on the modulation frequency or other factors. Further processing could be performed in signal module 473, such as intermediate frequency (IF) signal processing, filtering, conditioning, or other signal processing. Signal module 473 would then transfer the processed electrical versions of the optical signals over link 425 to ADC 416.

Tissue interface 440 is configured to couple with finger 450 and provide optical mating between optical links 431-434 and tissue of finger 450. Further elements could be included in tissue interface 440, such as a clamp, spring, band, adhesive, elastic sleeve, or other elements to couple the pad portion tightly to finger 450. Tissue interface 440 may be comprised of plastic, foam, rubber, glass, metal, adhesive, or some other material, including combinations thereof.

Optical cable 430 includes individual signaling links in this example, namely links 431-434. In this example, link 431 is a first input optical link, link 432 is second input optical link, link 433 is a reference input optical link, and link 434 is a measurement optical link. Each of links 431-434 could comprise individual optical fibers. Optical cable 430 could include a sheath or loom to bundle each of links 431-434 together for convenience. One end of each of optical links 431-434 terminates and optically mates with an associated component of measurement device 401, and the other end of each of optical links 431-434 is configured to terminate in tissue interface 440 and emit light into finger 450 or receive light from finger 450. When optical fibers are employed in optical cable 430, each optical fiber comprises an optical waveguide, such as a glass or polymer fiber, for transmission of light therein, and could include multimode fiber (MMF) or single mode fiber (SMF) materials.

In operation, tissue interface 440 will be coupled to finger 450 of a patient undergoing measurement of physiological parameters. A user will instruct through user interface 417 to initiate a measurement process with measurement device 401. These user instructions will be transferred over link 427 for receipt by processing module 410. In response, processing module 410 will initiate instructions and control signaling over link 421 for signal generator 411 to generate signals for lasers 412-414. Lasers 412-414 will emit optical signals on associated optical links 431-433 according to the inputs received over links 422-424. In this example, links 422-424 each employ electrical signaling and lasers 412-414 each interpret the electrical signaling for output as an optical signal.

In some examples, photon density wave (PDW) techniques are employed within finger 450. To establish a PDW, signal generator 411 first generates a high-frequency modulated drive signals for lasers 412-414. These high-frequency modulated signals could each comprise an amplitude modulated signal at one gigahertz or higher. It should be understood that lower modulation frequencies could be employed. Lasers 412-414 each receive this modulated signal over associated links 422-424 and in response, emit a corresponding optical signal modulated according to the received modulated signals. Thus, although lasers 412-414 each emit an optical signal of a certain wavelength, these optical signals are further modulated at a high rate, according to the received signal over links 422-424.

In tissue interface 440, the optical links are shown routed to varying locations. First input link 431 is routed to a first location, second input link 432 is routed to a second location, reference input link 433 is routed to a third location, and measurement link 434 is routed to a similar location as reference input link 433. These routes and depths are merely exemplary in this example, and typically are not stacked in a vertical fashion as shown in FIG. 4. Accordingly, first input link 431, second input link 432, and reference input link 433 will each have emission points for associated optical signals at the locations shown. Measurement link 434 will receive the optical signals at the third location, as indicated by the “waves” arrows in FIG. 4.

Since the termination point of reference input link 433 is located adjacent or proximate to the termination point of measurement link 434, any optical signal emitted by reference input link 433 would only propagate a short distance for receipt into measurement link 434. In examples where separate optical fibers are employed, the optical fiber associated with reference input link 433 and the optical fiber associated with measurement link 434 would terminate at the same or similar location within tissue interface 440. Likewise, any optical signal emitted by first input link 431 or second input link 432 would have propagated through a deeper and more substantial portion of finger 450 than an optical signal emitted by reference link 433.

The optical signals received by link 434 are transferred over optical cable 430 for receipt by detection and separation module 415. Detection and separation module 415 includes optical detection elements which convert the received optical signals to corresponding electrical representations. In this example, multiple optical signals could be carried over measurement link 434. For example, a multiplexing configuration could be employed to share a single photodetector or measurement link 434 among multiple input optical signals. It should be understood that the detection of optical signals and translation into electrical signals could occur prior to or subsequent from the separation of multiplexed signals by detection and separation module 415.

In a first example multiplexing configuration, wavelength division multiplexing (WDM) is employed. Each of lasers 412-414 would be configured to simultaneously emit optical signals at a different wavelength of light over respective links 431-434. The different wavelengths emitted by lasers 412-414 would all be proximate to a target wavelength, such as the isosbestic point of hemoglobin, but would also be separated by suitable spectral guard bands to allow subsequent optical signal separation by detection and separation module 415. In PDW examples, each of lasers 412-414 would receive a similar modulation signal over respective links 422-424 and modulate the associated wavelength of light according to the modulation signals. Measurement link 434 would then receive all wavelengths of light as transmitted by lasers 412-414, and detection and separation module 415 would be configured to detect these various wavelengths. Detection and separation module 415 would separate the various wavelengths of light carrying each optical signal. In some examples, detection and separation module 415 receives and splits, filters, and separates the optical signals received based on wavelength. For example, three different wavelengths could be received over measurement link 434 due to use of three lasers 412-414. Detection and separation module 415 would detect the optical signals for each wavelength and separate optical signals originally introduced by lasers 412-414 based on wavelength. Thus, although three input links are employed in FIG. 4, only one output link is necessary to detect the optical signals introduced into finger 450 by the three input links.

In a second example multiplexing configuration, frequency domain multiplexing (FDM) is employed. In FDM, in conjunction with PDW techniques, different PDW modulation frequencies are used over each of links 422-424 to drive each of lasers 412-414. The modulation signals could be gigahertz-range frequencies separated by suitable guard bands, such as 10 kilohertz, to provide electronic separation over links 422-424 as well as optical separation once emitted by the associated laser. Each of lasers 412-414 would be configured to simultaneously emit optical signals over respective links 431-434 at the same wavelength but modulated according to the different modulation frequencies. Measurement link 434 would then receive all the optical signals as transmitted by lasers 412-414, and detection and separation module 415 would be configured to detect the optical signals. Detection and separation module 415 would filter the optical signals according to the different modulation frequencies. In some examples, detection and separation module 415 receives and splits, filters, and separates the optical signals received based on modulation frequency. Various filters could be used, including band pass filters. As another example, three different optical signals could be received over measurement link 434 due to use of three lasers 412-414. Detection and separation module 415 would detect the optical signals and separate the optical signals originally introduced by lasers 412-414 based on modulation frequencies. Thus, although three input links are employed in FIG. 4, only one output link is necessary to detect the optical signals introduced into finger 450 by the three input links.

The multiplexing configuration could include time domain multiplexing (TDM), where optical signals transferred over each of links 431-433 are alternately applied to finger 450 in a time-staggered fashion. Other configurations could be employed, such as code-division multiplexing (CDM), where additional code-based modulation on the optical signals is employed to create code-separated channels. Frequency multiplexing, frequency hopping, chirping, or spread spectrum techniques could also be employed.

ADC 416 would then receive over link 425 the electrical signals as determined and separated by detection and separation module 415. ADC 416 converts these signals into a digital format for delivery to processing module 410 over link 426. Processing module 410 processes the received information to determine characteristics of the received signals as well as identify values of physiological parameters based on the received signals, such as the heart rate and the oxygen saturation of hemoglobin. Processing module 410 could transfer these values of the physiological parameters to user interface 417 over link 427 for display to a user.

Advantageously, in FIG. 4, measurement link 434 receives optical signals emitted by reference input link 433 without significant tissue penetration of finger 450. This configuration also minimizes phase delay and amplitude errors introduced by long optical links since reference input link 433 is routed along with measurement link 433 as well as with first input link 431 and second input link 432. A bend in cable 430 that is caused by patient motion or other physical movement would affect first input link 431, second input link 432, reference input link 433, and measurement link 434 in a similar manner. Thus, comparisons between reference link 433 and first input link 431 or second input link 432 would tend to compensate for errors introduced by long or bent optical links.

In further examples of system 300 in FIG. 3 and system 400 in FIG. 4, the received signals detected by the associated detector could be downconverted to an intermediate frequency (IF) using common communication system tuner techniques, such as heterodyning. A combined programmable gain block and downconversion block may be found in many commodity components and devices. The baseband or IF signals could then be directly digitized and transferred to the processing module which calculates amplitude and phase delays instead of discrete phase and amplitude detector circuitry. A wider range of input phase relationships could be handled in this manner. In IF examples, an ADC must have sufficient bandwidth to sample the IF rather than the baseband phase and amplitude signals, and detector 313 could be comprise by a mixer or radio tuner circuit. Downconverting to IF and digitizing can have advantages over some example phase and amplitude detectors, such as an AD8302, because certain phase and amplitude detector circuitry may not perform well at certain phase differences between the input and reference signal and require more precise control of phase and amplitude inputs. Signal modules 362, 463, or 473 could perform this IF processing.

Also, as seen in FIGS. 3 and 4, the configuration of the tissue interface is for a reflectance-based measurement, where emitted and received signals are coupled to the same side of a tissue portion and a reflection of optical signals is the dominant detection pathway. In other examples, a transmission-based measurement could be employed, where emitted signals are applied on an opposite side of or significantly displaced along the tissue as a detector and transmission of optical signals is the dominant detection pathway. A combination of reflectance and transmission could be employed.

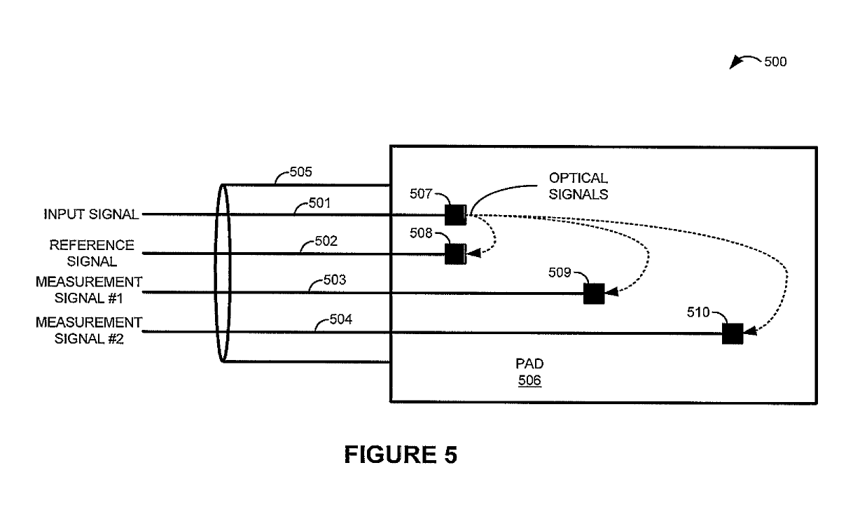

FIG. 5 illustrates tissue interface assembly 500 that emits optical signals to tissue and receives a reference optical signal and two measurement optical signals from the tissue. Tissue interface assembly 500 is an example of pad 160, tissue interface 340, tissue interface 440, kayak 710, or pad 810, although these may use other configurations. Tissue interface assembly 500 comprises pad 506 that is coupled to fiber optic cable 505. Pad 506 may be comprised of a rubber, foam, plastic, metal, or some other material, including combinations thereof. Pad 506 includes optical signal emission point 507 and optical signal collection points 508-510. In some examples, emission and collection points 507-510 may include optical interface elements such as prisms, mirrors, diffusers, and the like to optically couple the associated optical fibers to the tissue under measurement. In other examples, emission and collection points 507-510 may comprise the ends of associated optical fibers oriented to face the tissue to optically couple the associated optical fibers to the tissue. A first surface of pad 506 is flatly contoured so tissue of a patient will make continuous contact with pad 506 to fully optically couple to emission and collection points 507-510.

Fiber optic cable 505 comprises optical fibers 501-504. Optical fiber 501 terminates at emission point 507. Optical fibers 502-504 terminate at respective collection points 508-510. Optical fibers 501-504 are coupled to pad 506 through channelized compression and/or an adhesive compound. Note that collection point 508 is adjacent to emission point 507. Collection point 509 is spaced at a first distance, such as 5-6 millimeters (mm), from emission point 507, and collection point 510 is spaced at a second distance, such as 10-12 mm, from emission point 507.

The optical signals are propagated by optical fiber 501 to emission point 507 where it is emitted toward the tissue. Collection point 508 collects the optical signals, and due to its adjacent position to emission point 507, collection point 508 receives the optical signals with little or no tissue penetration, and thus little or no influence on optical signal characteristics by the tissue. Optical fiber 502 propagates the received optical signals from collection point 508 for subsequent detection and processing as a reference signal. Due to associated larger distances from emission point 507, collection points 509-510 each receive optical signals that have moderate-to-deep tissue penetration. Optical fiber 503 propagates first received optical signals from collection point 509 which have optical signal characteristics, such as a phase and amplitude, affected by a first amount of tissue penetration. Optical fiber 504 propagates second received optical signals from collection point 510 which have optical signal characteristics affected by a second amount of tissue penetration. In this example, the propagation or scattering of the optical signals emitted at emission point 507 is minimal at collection point 508, an intermediate amount at collection point 509, and a largest amount at collection point 510.

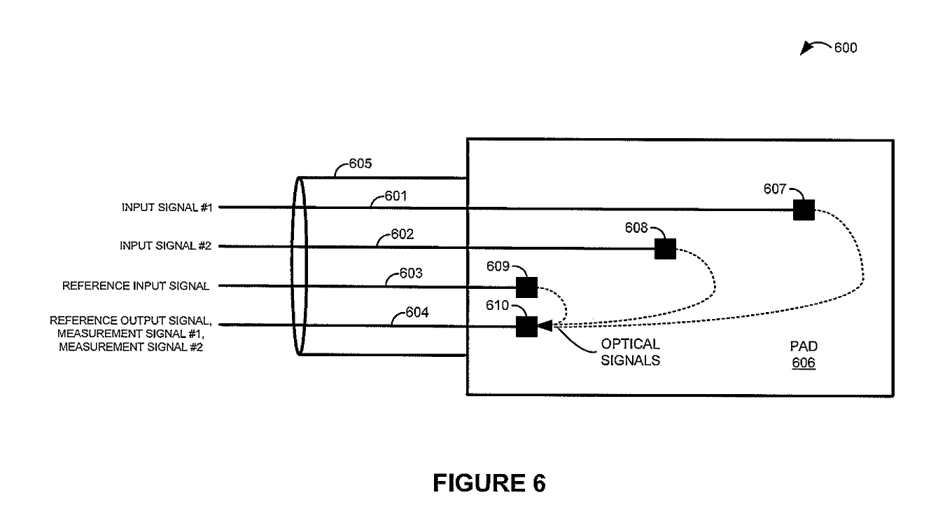

FIG. 6 illustrates tissue interface assembly 600 that emits two input optical signals and a reference input optical signal to tissue and receives a reference output optical signal and two measurement optical signals from the tissue. Tissue interface assembly 600 is an example of pad 160, tissue interface 340, tissue interface 440, kayak 710, or pad 810, although these may use other configurations. Tissue interface assembly 600 comprises pad 606 that is coupled to fiber optic cable 605. Pad 606 may be comprised of rubber, foam, plastic, metal, or some other material, including combinations thereof. Pad 606 includes optical signal emission points 607-609 and optical signal collection point 610. In some examples, emission and collection points 607-610 may include optical interface elements such as prisms, mirrors, diffusers, and the like to optically couple the associated optical fibers to the tissue under measurement. In other examples, emission and collection points 607-610 may comprise the ends of the associated optical fibers oriented to face the tissue to optically couple the associated optical fibers to the tissue. A first surface of pad 606 is flatly contoured, so tissue of a patient will make continuous contact with pad 606 to fully optically couple to emission and collection points 607-610.

Fiber optic cable 605 comprises optical fibers 601-604. Optical fibers 601-603 terminate at respective emission points 607-609. Optical fiber 604 terminates at collection point 610. Optical fibers 601-604 are coupled to pad 606 through channelized compression and/or an adhesive compound. Note that emission point 609 is adjacent to collection point 610. Emission point 608 is spaced at a first distance, such as 5-6 mm, from collection point 610, and emission point 607 is spaced at a second distance, such as 10-12 mm, from collection point 610.

The first input optical signal is propagated by optical fiber 601 to emission point 607 where it is emitted toward the tissue. Due to its large distance from emission point 607, collection point 610 receives optical signals associated with the first input optical signal after a first amount of optical signal propagation through the tissue, such as a deep tissue penetration. Optical fiber 604 propagates a first measurement optical signal comprised of received optical signals from collection point 610 which will have optical signal characteristics, such as a phase and amplitude, affected according to the first amount of optical signal propagation.

The second input optical signal is propagated by optical fiber 602 to emission point 608 where it is emitted toward the tissue. Due to its moderate distance from emission point 608, collection point 610 receives optical signals associated with the second input optical signal after a second amount of optical signal propagation through the tissue, such as a moderate tissue penetration. Optical fiber 604 propagates a second measurement optical signal comprised of received optical signals from collection point 610 which will have optical signal characteristics affected according to the second amount of optical signal propagation.

The reference input optical signal is propagated by optical fiber 603 to emission point 609 where it is emitted toward the tissue. Since collection point 610 is adjacent to emission point 609, collection point 610 receives optical signals associated with the reference input signal after a third minimal amount of optical signal propagation through the tissue, such as little or no tissue penetration. Optical fiber 604 propagates a reference optical signal comprised of received optical signals from collection point 610 which will have optical signal characteristics minimally affected or not affected according to the third amount of optical signal propagation.

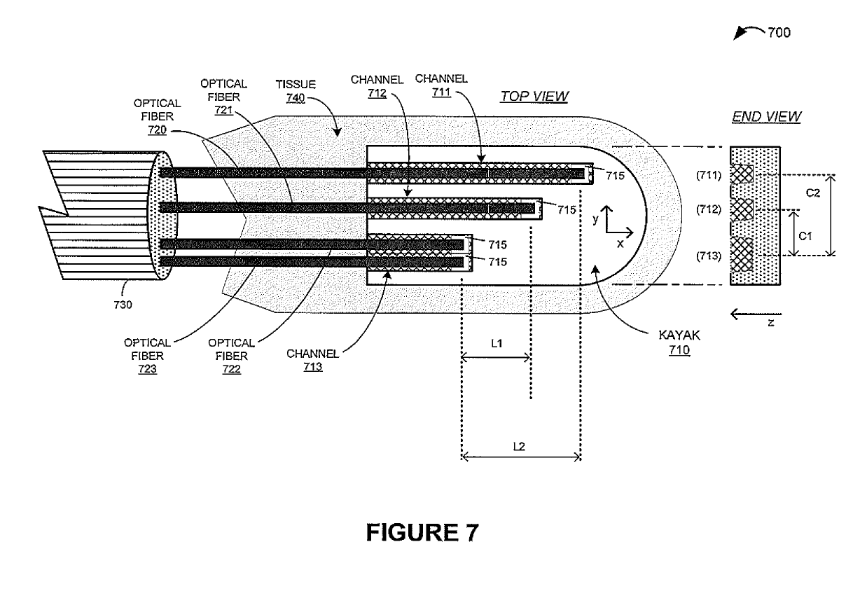

FIG. 7 is a system diagram illustrating tissue interface assembly 700. Tissue interface assembly 700 includes kayak 710 and optical cable 730. Kayak 710 is an example of pad 160, tissue interface 340, tissue interface 440, pad 506, or pad 606, although these may use different configurations. Kayak 710 is coupled to tissue 740 in this example. Tissue 740 could comprise any tissue described herein, such as a finger. Optical cable 730 comprises several optical fibers, namely optical fibers 720-723, for carrying optical signals to and from kayak 710.

In FIG. 7, several axes are shown for reference purposes. For the top view, a ‘y’ axis is shown relative to the ‘up-down’ page orientation and an ‘x’ axis is shown relative to the ‘left-right’ page orientation. For the end view, a ‘z’ axis is shown in the side view as a thickness of kayak 710.

Kayak 710 comprises a surface for contacting tissue 740. In operation, kayak 710 will lay coincident on tissue 740. In this example, kayak 710 is configured in a reflectance-type measurement configuration. Kayak 710 also comprises several channels 711-713 for routing optical fibers 720-723 to the locations shown. Each channel is positioned at a specific channel location in the ‘y’ direction, namely C1 and C2 indicating centerlines for the channel locations relative to channel 713. The depth of each channel 711-713 in the ‘z’ direction is determined by the thickness of kayak 710, and the size of each optical fiber or optical interface elements, among other considerations. Each channel is routed to a certain length within kayak 710 in the ‘x’ direction, namely L1 and L2 indicating lengths of each channel within kayak 710 relative to channel 713. In this example, channel 713 is used as a baseline for the other dimensions, although other dimensional configurations could be employed. In typical examples, kayak 710 is colored dark to minimize optical reflection and stray light. In some examples, kayak 710 is coated or anodized to a dark color, while in other examples kayak 710 is composed of a dark material such as plastic with injected dark pigment.

In this example, optical fiber 723 is an input optical fiber for introducing optical signals into tissue 740. The other optical fibers terminate at locations relative to the input optical fiber 723. Specifically, the termination point of reference output optical fiber 722 is located adjacent to the termination point of input optical fiber 723, the termination point of first measurement optical fiber 721 is located a first distance from the termination point of input optical fiber 723, and the termination point of second measurement optical fiber 720 is location a second distance from the termination point of input optical fiber 723. Typical spacing between the input optical fiber termination point and the measurement optical fiber termination points are 5-10 mm for arterial-based tissue measurements, and 30-40 mm for cerebral-based tissue measurements. In this example, the input optical fiber 723 termination point is 5 mm (diagonally) from the first measurement optical fiber 721 termination point, and the input optical fiber 723 termination point is 10 min (diagonally) from the second measurement optical fiber 720 termination point. Thus, in this example, a staggered spacing arrangement of the channels and optical fibers is employed. Advantageously, this spacing arrangement allows the optical fibers to be aligned generally parallel within kayak 710 and thus optical cable 730 is aligned along the length of tissue 740. This parallel configuration allows for greater repeatability in measurement and consistent coupling of kayak 710 to tissue 740 by reducing perpendicular stresses and forces on the optical fibers and kayak 710. Although specific spacing and location dimensions are given herein, it should be understood that the dimensions may vary. Also, although tissue interface assembly 700 includes two measurement optical signals and associated optical fibers, a different number of measurement optical signals and associated optical fibers could be employed.

Kayak 710 also includes optical interface elements 715. Since the optical fibers transport optical signals parallel to the surface of tissue 740, a 90 degree optical turn must be established to properly introduce the optical signals into tissue 740 or to properly detect optical signals from tissue 740. Each optical interface element 715 could comprise a prism, lens, minor, diffuser, and the like, to optically couple the associated optical fibers to the tissue under measurement. The optical interface elements 715 could each be adhered to the associated optical fiber end, such as with glue or other adhesive.

The interface between input optical fiber 723 and reference output optical fiber 722 could comprise air, space, or a material, including combinations thereof. In many examples, it is desirable to leak some portion of the optical signal, such as light, out of the fiber-to-fiber interface between input optical fiber 723 and reference output optical fiber 722 to allow reference output optical fiber 722 to capture some of the optical signal emitted by input optical fiber 723. This leak could be performed by fiber-couplers, a weak reflection off an optical interface at the output of the fiber, or other similar configurations. The light leaked out of input optical fiber 723 for reference output optical fiber 722 could then be scattered by a second material. The types of materials for the second material could comprise scattering media such as Teflon, PVCs, (i.e. light-colored/white, diffuse/“milky” plastics), cloudy glasses, thin glass sheets with both surfaces etched as to diffuse the light, holographic scatterers, or similar materials. Additionally, this material could comprise a diffuser shim inserted between the input optical fiber 723 and reference output optical fiber 722 to reduce the dependency of reference output optical fiber 722 on pressure of the surface portion of kayak 710 on tissue 740 and to randomize optical reflection modes between input optical fiber 723 and reference output optical fiber 722. In further examples, reference output optical fiber 722 could receive optical signals through tissue 740, such as discussed herein for minimal penetration or propagation of reference optical signals. In these minimal propagation examples, reference output optical fiber 722 would be positioned adjacent to input optical fiber 723, and terminate at a similar location, but instead of receiving optical signals through a direct or leaky fiber-to-fiber interface, would receive optical signals through a small portion of tissue 740.

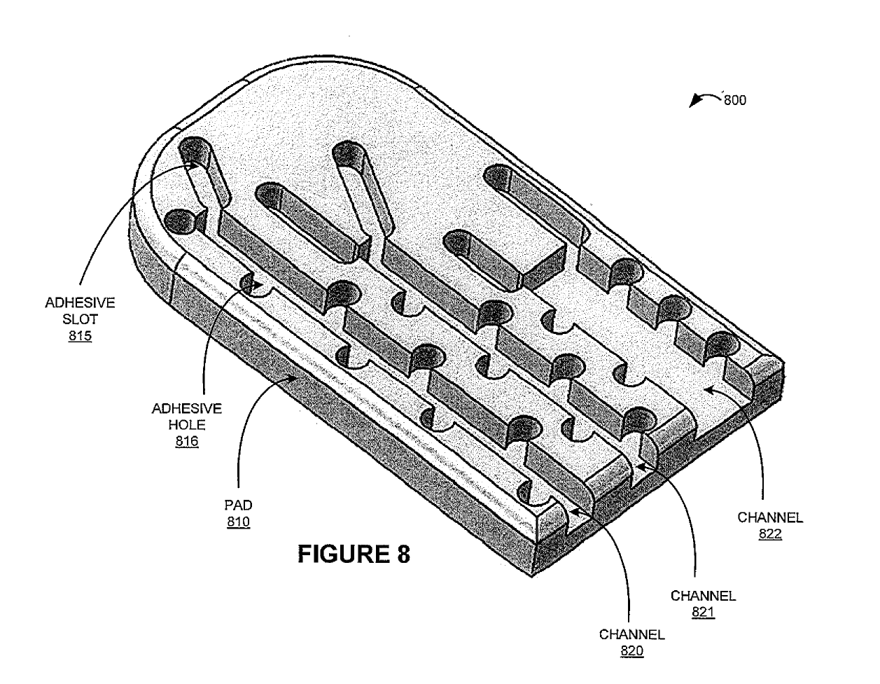

FIG. 8 is an oblique view diagram illustrating tissue interface pad 800. Tissue interface pad 800 is an example of pad 160, tissue interface 340, tissue interface 440, pad 506, pad 606, or kayak 710, although these may use different configurations. Tissue interface assembly includes pad 810 which has several channels and adhesive elements. Channels 820-822 comprise grooves formed into pad 810 for routing optical fibers to various termination points as shown. Adhesive slots 815 and adhesive holes 816 are distributed about each of channels 820-822.

Adhesive slots 815 and adhesive holes 816 are used to inject adhesive, such as glue, into and around each channel to securely couple each optical fiber and interface elements into the associated channel. Typically, an optical fiber would be inserted into a channel, and adhesive would be injected, such as by a needle injector, into the associated adhesive slots 815 and adhesive holes 816 until enough adhesive is applied to hold the optical fiber. A curing process could then be performed to cure the adhesive. The adhesive could include an ultraviolet (UV) cured adhesive or other accelerated-curing adhesives. In further examples, pad 810 could act as an in-situ alignment guide for optical fibers, where a fixture with soft tip set screws is employed to hold individual optical fibers in place radially (after rotating the optical fiber to a desired position), followed by an application of adhesive. This fixture ensures the desired rotation between the fiber and associated optical interface element, such as a prism, is established by holding the various optical elements in place until the adhesive is cured. After curing, the fixture with set screws could then be removed.

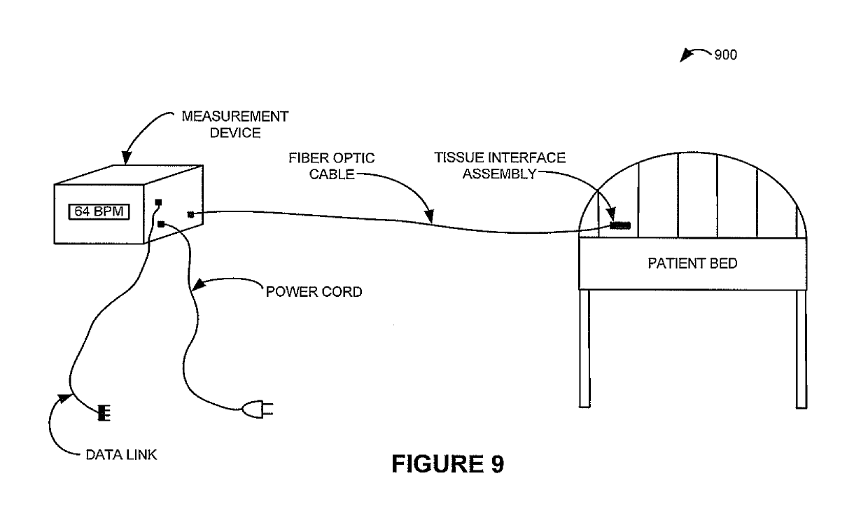

FIG. 9 illustrates system 900 in a typical operating environment. System 900 includes a measurement device that is coupled to a tissue interface assembly by a flexible fiber optic cable. Although the patient is not shown in the patient bed for clarity, the tissue interface assembly is comfortably strapped to tissue of the patient, such as a finger, toe, earlobe, forehead, or other tissue portion. The flexibility of the fiber optic cable allows the patient some freedom of movement and allows the measurement device to be placed away from the patient bed. The measurement device houses the optical signal transmitters, optical signal receivers, and processing elements such as discussed herein. The measurement device also has a display to directly indicate the physiological parameters, such as a blood metrics, for the patient. In this example, a heart rate in Beats Per Minute (BPM) is shown. The measurement device also has a data link to transfer the physiological parameters to other systems for analysis, reporting, or storage, and could comprise signaling as described for link 115 in FIG. 1. The measurement device also has a power cord to supply power.

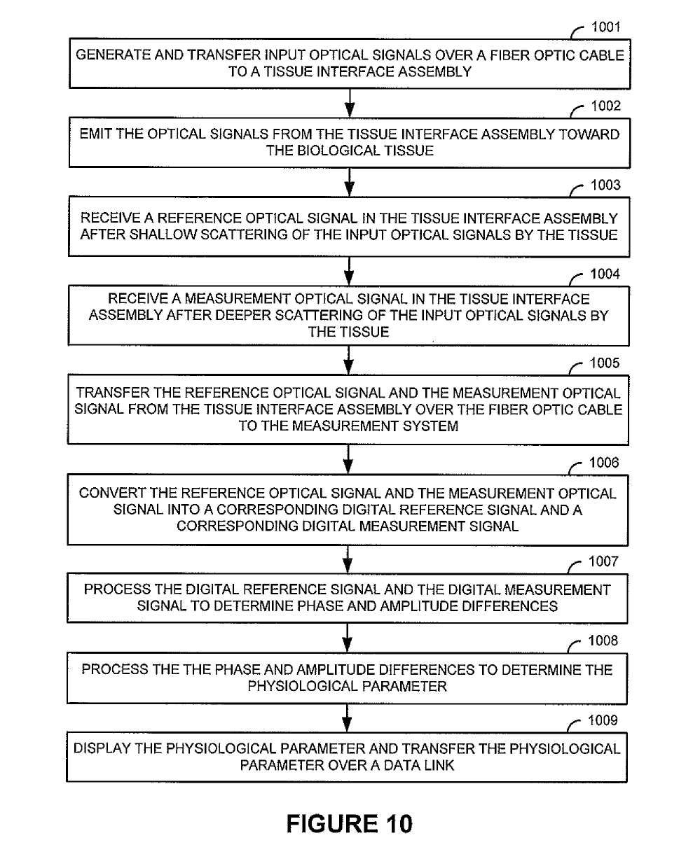

FIG. 10 illustrates the operation of a system to analyze biological tissue, such as described in the embodiments herein. A transceiver portion generates input optical signals and transfers the input optical signals over a fiber optic cable to a tissue interface assembly (1001). The tissue interface assembly receives the input optical signals from the fiber optic cable and emits the input optical signals toward the biological tissue (1002), where the input optical signals are scattered by the tissue. The tissue interface assembly receives a reference optical signal from the relatively shallow scattering of input optical signal that does not introduce significant phase and amplitude differences (1003). The tissue interface assembly receives a measurement optical signal from the relatively deep scattering of the input optical signal that does introduce significant phase and amplitude differences (1004). The tissue interface assembly transfers the reference optical signal and the measurement optical signal over the fiber optic cable to the transceiver portion (1005).

The transceiver portion receives the reference optical signal from the fiber optic cable and converts the reference optical signal into a digital reference signal (1006). The transceiver receives the measurement optical signal from the fiber optic cable and converts the measurement optical signal into a digital measurement signal (1006). A processing portion of the measurement system processes the digital reference signal and the digital measurement signal to determine phase and amplitude differences between the optical signals that were introduced into the tissue (1007). The processing portion processes the phase and amplitude differences that were introduced by scattering or propagation in the tissue to determine a physiological parameter for the tissue, such as the heart rate or the oxygen saturation of hemoglobin (1008). The processing portion then drives a user interface, such as a display, with the physiological parameter and transfers the physiological parameter over a data link (1009).

In some alternative examples to the above process in FIG. 10, the transceiver portion also generates and transfers a reference input optical signal over the fiber optic cable to the tissue interface assembly. The tissue interface assembly receives the reference input optical signal from the fiber optic cable and emits the reference input optical signal into the biological tissue. The tissue interface assembly receives a reference output optical signal after the relatively shallow scattering of the reference input optical signal.

The included descriptions and drawings depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the invention. Those skilled in the art will also appreciate that the features described above can be combined in various ways to form multiple embodiments. As a result, the invention is not limited to the specific embodiments described above, but only by the claims and their equivalents.

What is claimed is:

1. A system to optically analyze tissue of a patient comprising:
a tissue interface assembly configured to emit an input optical signal into the tissue, receive a reference optical signal and a measurement optical signal from the tissue, and transfer the reference optical signal and the measurement optical signal to an optical link;
the optical link configured to transfer the reference optical signal and the measurement optical signal; and
a transceiver configured to receive the reference optical signal and the measurement optical signal and process the reference optical signal and the measurement optical signal to identify a value of a physiological parameter of the patient.

2. The system of claim 1 further comprising:
the transceiver configured to convert the reference optical signal into a digital reference signal and to receive and convert the measurement optical signal into a digital measurement signal; and
a digital processor configured to process the digital measurement signal and the digital reference signal to determine a phase delay between the digital measurement signal and the digital reference signal and process the phase delay to identify the physiological parameter of the patient.

3. The system of claim 1 wherein:
the transceiver comprises a laser configured to generate the input optical signal;
the optical link comprises a first optical fiber configured to transfer the input optical signal from the transceiver to the tissue interface assembly, a second optical fiber configured to transfer the reference optical signal from the tissue interface assembly to the transceiver, and a third optical fiber configured to transfer the measurement optical signal from the tissue interface assembly to the transceiver.

4. The system of claim 1 wherein the optical link comprises a flexible fiber optic cable.

5. The system of claim 1 wherein:
the transceiver comprises a first laser configured to generate the input optical signal and a second laser configured to generate an input reference optical signal;
the optical link comprises a first optical fiber configured to transfer the input optical signal to the tissue interface assembly and a second optical fiber configured to transfer the input reference optical signal to the tissue interface assembly;
the tissue interface assembly is configured to receive the input reference optical signal from the optical link and emit the input reference optical signal into the tissue;
the optical link comprises a third optical fiber configured to transfer the reference optical signal and the measurement optical signal from the tissue interface assembly to the transceiver; and
the transceiver comprises a detector portion configured to detect the reference optical signal and the measurement optical signal.

6. The system of claim 5, further comprising:
the transceiver configured to convert the reference optical signal into a digital reference signal and to receive and convert the measurement optical signal into a digital measurement signal; and
a digital processor configured to process the digital measurement signal and the digital reference signal to determine a phase delay between the digital measurement signal and the digital reference signal and process the phase delay to identify the physiological parameter of the patient.

7. The system of claim 1 wherein the transceiver comprises:
a first laser and a second laser;
a signal generator configured to generate a first modulation signal to drive the first laser to emit the input optical signal modulated at a first frequency;
the signal generator configured to generate a second modulation signal to drive the second laser to emit the input reference optical signal modulated at a second frequency.

8. The system of claim 7 wherein the transceiver further comprises:
an optical detector configured to detect the measurement optical signal and the reference optical signal and transfer a corresponding electronic detection signal;
a filter configured to receive and filter the electronic detection signal to transfer an electronic measurement signal modulated at the first frequency and to transfer an electronic reference signal modulated at the second frequency;
a signal module configured to modulate the electronic measurement signal from the first frequency to an intermediate frequency and to modulate the electronic reference signal from the second frequency to the intermediate frequency;
an analog-to-digital conversion system configured to convert the electronic reference signal at the intermediate frequency into a digital reference signal and to convert the electronic measurement signal at the intermediate frequency into a digital measurement signal; and
a digital processor configured to process the digital measurement signal and the digital reference signal to determine a phase delay between the digital measurement signal and the digital reference signal and process the phase delay to identify the physiological parameter of the patient.

9. The system of claim 8 wherein:
the optical link comprises a single optical fiber configured to transfer the reference optical signal and the measurement optical signal from the tissue interface assembly to the transceiver.

10. The system of claim 8 wherein:
the optical detector comprises a single photodetector configured to detect both the measurement optical signal and to detect the reference optical signal.

11. The system of claim 1 further comprising:
a scattering element configured to receive the input optical signal transfer the input optical signal as a scattered optical signal, wherein the reference optical signal comprises the scattered optical signal.

12. The system of claim 1 wherein:
the transceiver comprises a first laser configured to emit the input optical signal at a first wavelength and a second laser configured to emit an input reference optical signal at a second wavelength;
the tissue interface assembly is configured to receive the input reference optical signal from the optical link and emit the input reference optical signal into the tissue;
the optical link comprises a single optical fiber configured to transfer the reference optical signal and the measurement optical signal from the tissue interface assembly to the transceiver.

13. The system of claim 12 wherein the optical link comprises another single optical fiber configured to transfer the input optical signal and the input reference optical signal from the transceiver to the tissue interface assembly.

14. The system of claim 12 wherein the transceiver comprises:
an optical filter configured to receive and filter the measurement optical signal and the reference optical signal from the optical link and configured to transfer the measurement optical signal to a first photodetector and to transfer the reference optical signal to a second photodetector;
the first photodetector configured to detect the measurement optical signal and transfer a corresponding electronic measurement signal;
the second photodetector configured to detect the reference optical signal and transfer a corresponding electronic reference signal;
a signal module configured to modulate the electronic measurement signal and the electronic reference signal to the intermediate frequency;
an analog-to-digital converter module configured to convert the electronic reference signal at the intermediate frequency into a digital reference signal and to convert the electronic measurement signal at the intermediate frequency into a digital measurement signal; and
a digital processor configured to process the digital measurement signal and the digital reference signal to determine a phase delay between the digital measurement signal and the digital reference signal and process the phase delay to determine physiological parameter for the tissue.

15. The system of claim 12 further comprising:
a scattering element configured to receive the input reference optical signal transfer the input reference optical signal as a scattered optical signal, wherein the reference optical signal comprises the scattered optical signal.

16. A system to optically analyze tissue of a patient comprising:
a tissue interface assembly configured to emit an input optical signal at a first wavelength and modulated at a first frequency into the tissue, receive a reference optical signal and a measurement optical signal from the tissue, and transfer the reference optical signal and the measurement optical signal to a fiber optic cable;
the fiber optic cable comprising a second optical fiber configured to transfer the reference optical signal and the measurement optical signal from the tissue interface assembly to the transceiver;
the transceiver configured to receive and convert the reference optical signal into a digital reference signal and to receive and convert the measurement optical signal into a digital measurement signal; and
a digital processor configured to process the digital measurement signal and the digital reference signal to determine a phase delay between the digital measurement signal and the digital reference signal and process the phase delay to determine physiological parameter for the tissue.

17. The system of claim 16 wherein:
the transceiver is configured to emit an input reference optical signal at the first wavelength and modulated at a second frequency;
the fiber optic cable comprises a third optical fiber configured to transfer the input reference optical signal from the transceiver to the tissue interface assembly;
the tissue interface assembly is configured to receive the input reference optical signal from the fiber optic cable and emit the input reference optical signal into the tissue.

18. The system of claim 16 wherein:
the transceiver is configured to emit an input reference optical signal at a second wavelength and modulated at the first frequency;
the first optical fiber is configured to transfer the input reference optical signal from the transceiver to the tissue interface assembly;
the tissue interface assembly is configured to receive the input reference optical signal from the fiber optic cable and emit the reference optical signal into the tissue.

19. A method of operating a system to analyze tissue of a patient, the method comprising:
generating an input optical signal and transferring the input optical signal over a fiber optic cable;
receiving the input optical signal from the fiber optic cable and emitting the input optical signal into the tissue;
receiving a reference optical signal and a measurement optical signal from the tissue and transferring the reference optical signal and the measurement optical signal over the fiber optic cable;
receiving the reference optical signal and the measurement optical signal from the fiber optic cable and converting the reference optical signal into a digital reference signal and converting the measurement optical signal into a digital measurement signal; and
processing the digital measurement signal and the digital reference signal to determine a phase delay between the digital measurement signal and the digital reference signal and processing the phase delay to determine physiological parameter for the tissue.

20. The method of claim 19 further comprising:
generating an input reference optical signal and transferring the input reference optical signal over the fiber optic cable; and
receiving the input reference optical from the fiber optic cable and emitting the input reference optical into the tissue.
